(19) United States
(12) Patent Application Publication
(10) Pub. No.: US 2017/0340848 A1
(43) Pub. Date: Nov. 30, 2017

(54) Support Device and Methods for Use

(71) Applicant: Mayar Medical, Inc., Gurnee, IL (US)

(72) Inventor: Sohrab Mayar, Gurnee, IL (US)

(21) Appl. No.: 15/606,196

(22) Filed: May 26, 2017

Related U.S. Application Data
(60) Provisional application No. 62/342,134, filed on May 26, 2016.
(60) Provisional application No. 62/382,416, filed on Sep. 1, 2016.

(51) Int. Cl. A61M 16/04; A61M 25/02

(52) U.S. Cl. CPC A61M 16/0488; A61M 16/0497; A61M 25/02; A61M 16/0465; A61M 2025/0206; A61M 2025/022; A61M 2210/0618; A61M 2210/0625; A61M 2025/0213

(57) ABSTRACT

The present disclosure provides a device including comprising: (i) a concave surface at a first end of the device, (ii) a conduit at a second end of the device, and (iii) a pair of side surfaces extending inwardly at an acute angle from the first end of the device to the second end of the device.

### RELATED APPLICATIONS

This application claims the benefit of priority to (i) U.S. Provisional Application No. 62/342,134 entitled “Support Device for Endotracheal and Tracheostomy Tubes,” filed May 26, 2016, and (ii) U.S. Provisional Application No. 62/382,416 entitled “Support Device and Methods for Use,” filed on Sep. 1, 2016, both of which are hereby incorporated by reference in their entirety.

### BACKGROUND

Unless otherwise indicated herein, the materials described in this section are not prior art to the claims in this application and are not admitted to be prior art by inclusion in this section.

Tracheal tubes are used to mechanically ventilate and oxygenate patients, provide a medical administration route for specialized gases, aerosolized medications, and/or provide a screening and therapeutic route for pulmonary procedures, visualizations, biopsies, etc. Examples of such tracheal tubes include tracheostomy or endotracheal tubes. These tubes have a proximal end (machine end, that emerges from the patient's mouth, nose, or through surgical opening in the neck/trachea) and a distal end (patient end located in trachea). Endotracheal tubes are usually secured in place with medical tape or an endotracheal tube support device. Although the medical tape or other commercially available devices secures the endotracheal tube at the proximal (patient's mouth), there is no additional support against the sheer weight of the ventilator circuit. Often, the pulling-down force of the ventilator circuit causes constant tugging on the adhesive portion of such endotracheal tube support devices causing skin break-downs and skin irritation. In addition, many patients require additional adaptors and ports added to the ventilator circuit for therapeutic reasons that increases the pulling-down force on the tube, causing constant tugging and even major emergencies such as accidental tube dislodging or unplanned extubation.

### SUMMARY

Thus, in a first aspect, a device is provided to provide support for a tracheal tube while maintaining a secure airway while the tracheal tube is positioned in a patient. The device includes (i) a concave surface at a first end of the device, (ii) a conduit at a second end of the device, and (iii) a pair of side surfaces extending inwardly at an acute angle from the first end of the device to the second end of the device.

In one embodiment of the first aspect, the edges of the first end of the device are rounded.

In another embodiment of the first aspect, a width of the device tapers from the first end to the second end, such that a width at the first end is greater than a width at the second end.

In another embodiment of the first aspect, the device further comprises a slot coupled to the conduit, wherein the slot is configured to open when a tube is positioned therein, and then snap back to close the conduit once the tube is in position in the conduit.

In another embodiment of the first aspect, the device further comprises a cutout positioned between the first end and the second end of the device. In one such embodiment, the device further comprises one or more slits positioned in one or more of an interior surface of the conduit, an interior surface of the cutout, or the pair of side surfaces.

In another embodiment of the first aspect, the device further comprises a removable, washable cover. In one such embodiment, the cover comprises a plastic or otherwise non-porous, waterproof, and sterile material, and wherein the device comprises memory foam, plastic foam, or popped polystyrene.

In another embodiment of the first aspect, the device comprises a non-porous foam material, such as closed-cell polyurethane or closed-cell polyethylene foam, or a crosslinked polyethylene foam.

In another embodiment of the first aspect, the pair of side surfaces comprises a first material, and wherein the concave surface comprises a second material that is different than the first material. In one such embodiment, the first material is more rigid than the second material.

In another embodiment of the first aspect, the device further comprises a coupling mechanism configured to position the device on the chest of the patient. In one embodiment, the the coupling mechanism comprises a strap fixed to a first side of the first end of the device, and a loop at a second side of the first end of the device. In another embodiment, the coupling mechanism comprises a hook fastener on at least a portion of the concave surface.

In another embodiment of the first aspect, a width of the device ranges from about 60 mm to about 175 mm, the height of the device ranges from about 50 mm to about 165 mm, and the depth of the device ranges from about 30 mm to about 110 mm.

In a second aspect, the a method is disclosed, comprising (i) positioning the device of the first aspect on a chest of a subject, and (ii) passing an endotracheal tube or a tracheostomy tube through the conduit at the second end of the device and into the subject.

In a third aspect, a device is provided, comprising: (i) a bottom surface, (ii) a first angled surface having a first end and a second end, (iii) a second angled surface having a first end and a second end, (iv) a first rounded surface coupling the bottom surface to the first end of the first angled surface, (v) a second rounded surface coupling the second end of the first angled surface to the first end of the second angled surface, and (vi) a third rounded surface coupling the second end of the second angled surface to the bottom surface.

In one embodiment of the third aspect, the device further comprises a through-hole having a longitudinal axis substantially parallel to a longitudinal axis of the second rounded surface.

In one embodiment of the third aspect, the first angled surface has a first angle with respect to the bottom surface, and wherein the second angled surface has a second angle with respect to the bottom surface that is greater than the first angle.

In one embodiment of the third aspect, a pair of side surfaces of the device comprise a first material, wherein the first and second angled surfaces comprise a second material that is different than the first material, and wherein the first material is more rigid than the second material.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1 illustrates a perspective view of a device, according to an example embodiment.

FIG. 2 illustrates a front view of the device, according to an example embodiment.

FIG. 3 illustrates another perspective view of the device, according to an example embodiment.

FIG. 4 illustrates another perspective view of the device, according to an example embodiment.

FIG. 5 illustrates a front view of a large version of the device, according to an example embodiment

FIG. 6 illustrates a front view of a small version of the device, according to an example embodiment.

FIG. 7 illustrates a perspective view of another version of the device, according to an example embodiment.

FIG. 8 illustrates a front view of the device of FIG. 7, according to an example embodiment.

FIG. 9 illustrates a side view of the device of FIG. 7, according to an example embodiment.

FIG. 10 illustrates a perspective view of another version of the device, according to an example embodiment.

FIG. 11 illustrates a front view of the device of FIG. 10, according to an example embodiment.

FIG. 12 illustrates a side view of the device of FIG. 10, according to an example embodiment.

FIG. 13 illustrates a perspective view of another version of the device, according to an example embodiment.

FIG. 14 illustrates a front view of the device of FIG. 13, according to an example embodiment.

FIG. 15 illustrates a perspective view of another version of the device, according to an example embodiment.

FIG. 16 illustrates a front view of the device of FIG. 15, according to an example embodiment.

FIG. 17 illustrates a perspective view of another version of the device, according to an example embodiment.

FIG. 18 illustrates a front view of the device of FIG. 17, according to an example embodiment.

FIG. 19 illustrates a perspective view of another version of the device, according to an example embodiment.

FIG. 20 illustrates a front view of the device of FIG. 19, according to an example embodiment.

FIG. 21 illustrates a perspective view of another version of the device, according to an example embodiment.

FIG. 22 illustrates a front view of the device of FIG. 21, according to an example embodiment.

FIG. 23 illustrates a perspective view of another version of the device, according to an example embodiment.

FIG. 24 illustrates a front view of the device of FIG. 23, according to an example embodiment.

FIG. 25 illustrates a perspective view of a device, according to an example embodiment.

FIG. 26 illustrates another perspective view of the device of FIG. 25, according to an example embodiment.

FIG. 27 illustrates a side view of the device of FIG. 25, according to an example embodiment.

### DETAILED DESCRIPTION

Example methods and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an example embodiment may include elements that are not illustrated in the Figures.

As used herein, with respect to measurements, “about” means+/−5%.

As used herein, “extubation” means the removal of the endotracheal tube or tracheostomy tube.

As used herein, “distal” means the end of a device (when in use) further away from the head of the patient and the term “proximal” means the portion of the device (when in use) nearer to the head of the patient.

As used herein, “hook fastener” means a material including a plurality of hooks configured to catch in loops of an opposing material.

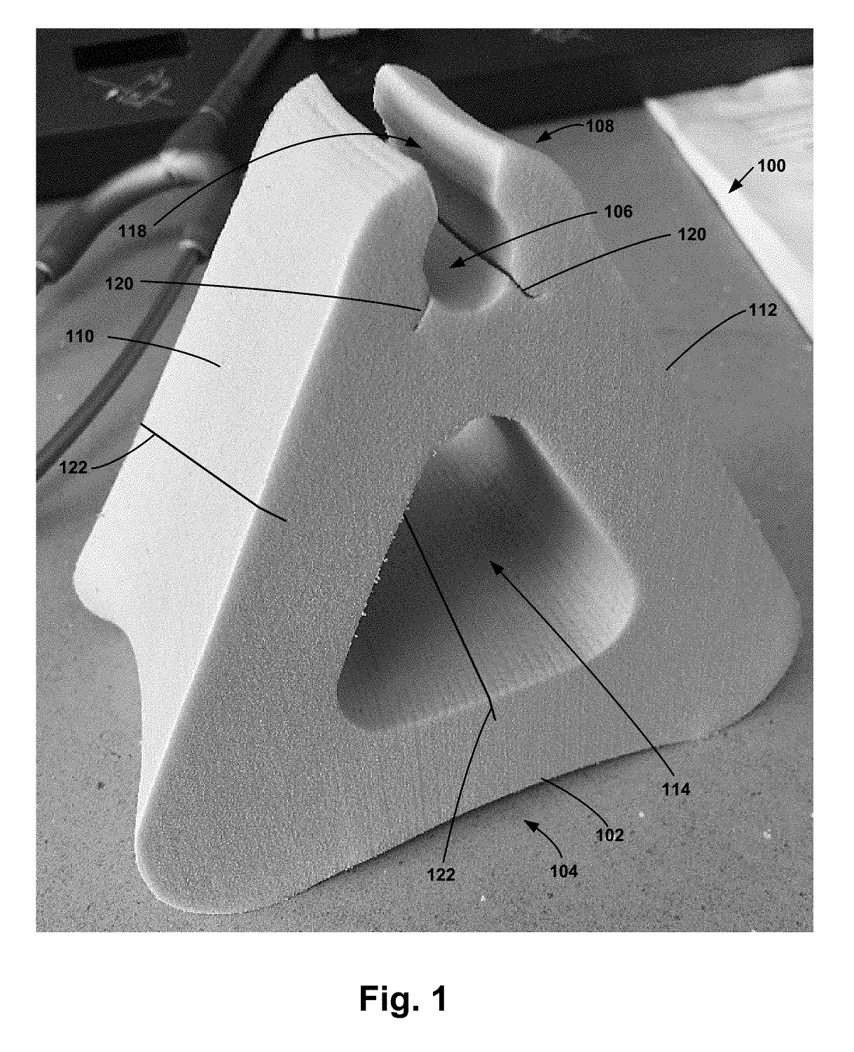

With reference to the Figures, FIGS. 1-4 illustrates an example device 100. In particular, the device 100 shown in FIGS. 1-4 may be used as an endotracheal tube or tracheostomy tube support device that helps support against the weight of the ventilator circuit. As such, the device 100 may help prevent unplanned endotracheal and tracheostomy tube extubations, and help support the endotracheal and tracheostomy tube holder device in critical patients with advanced airway or endotracheal tube for ventilation.

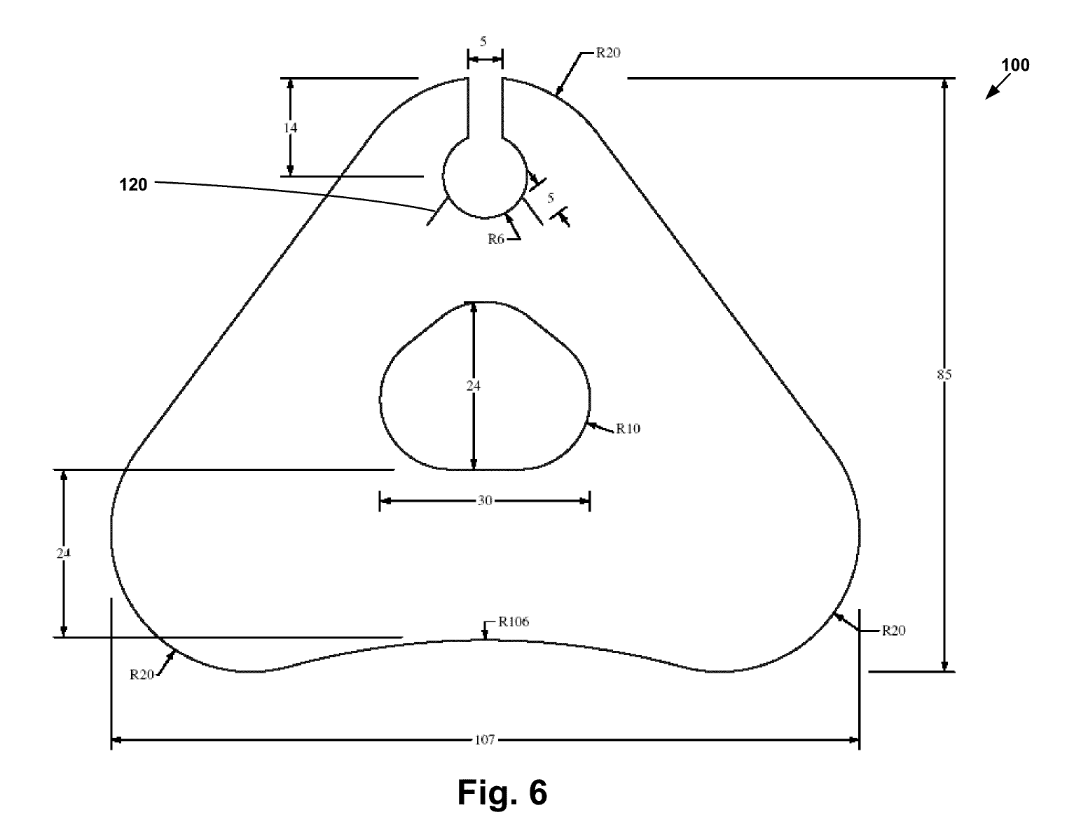

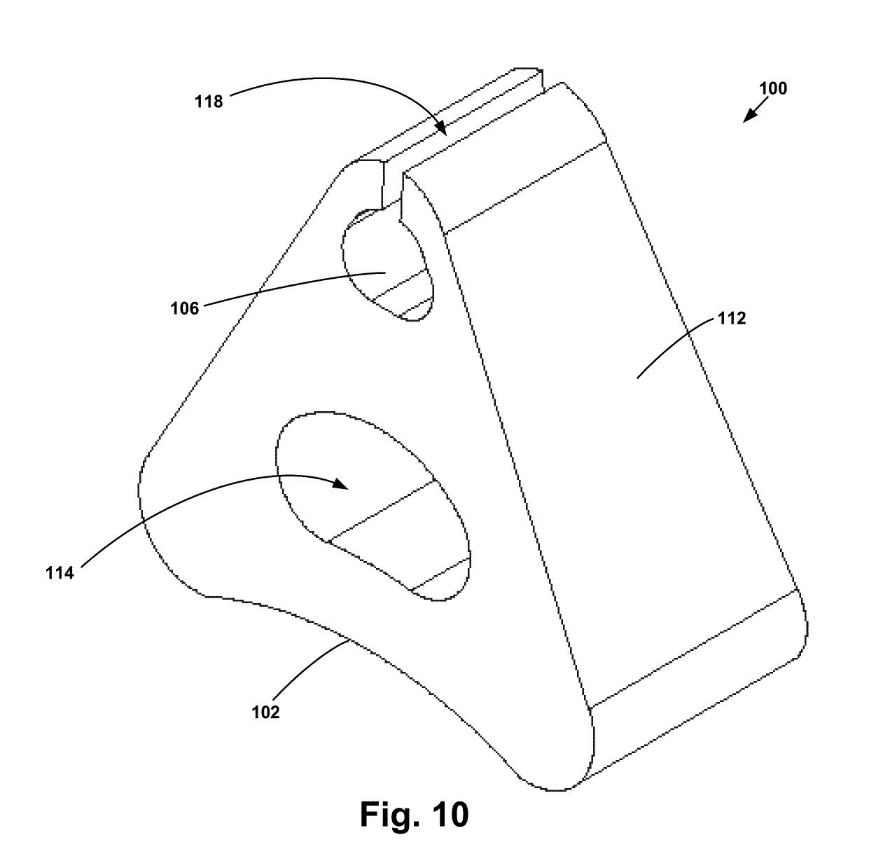

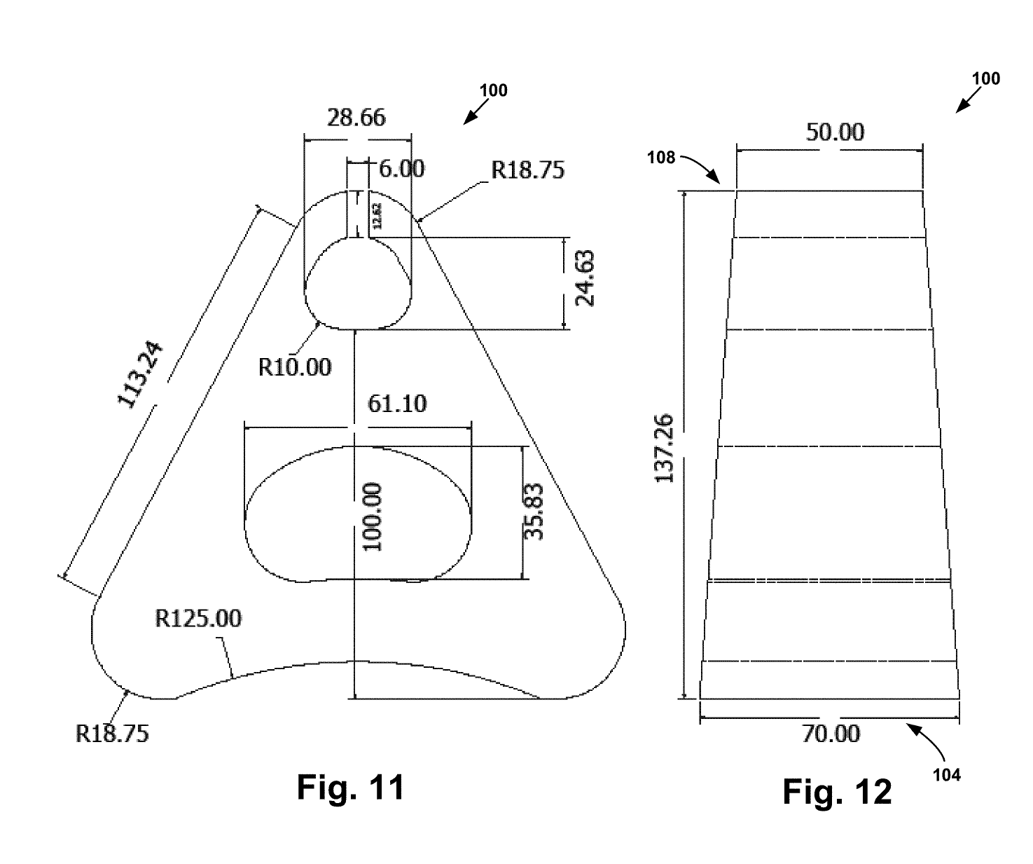

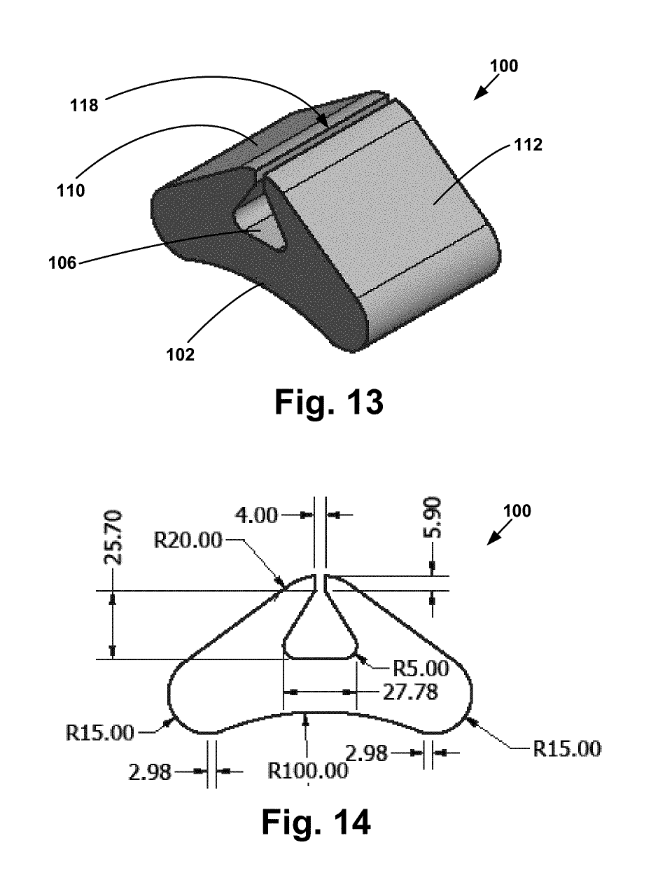

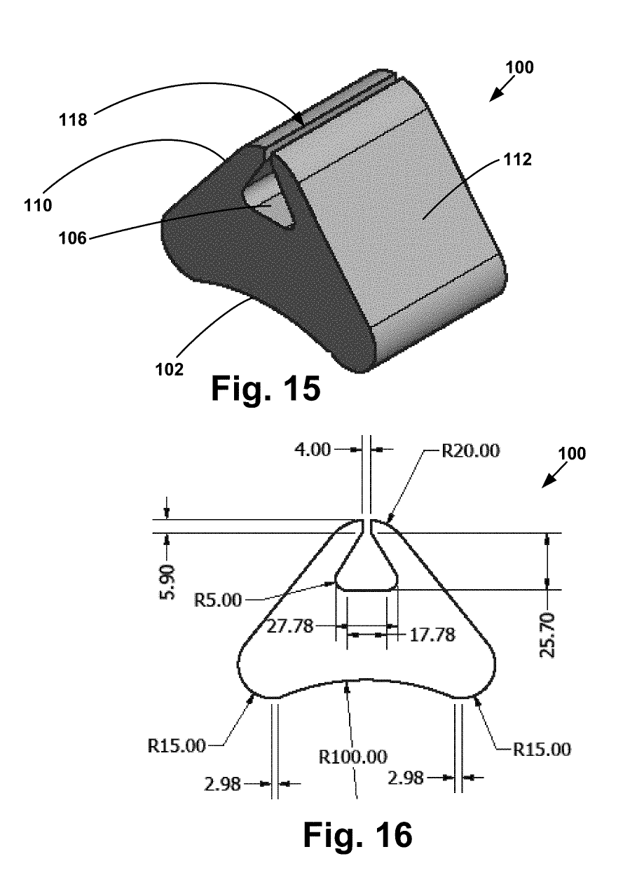

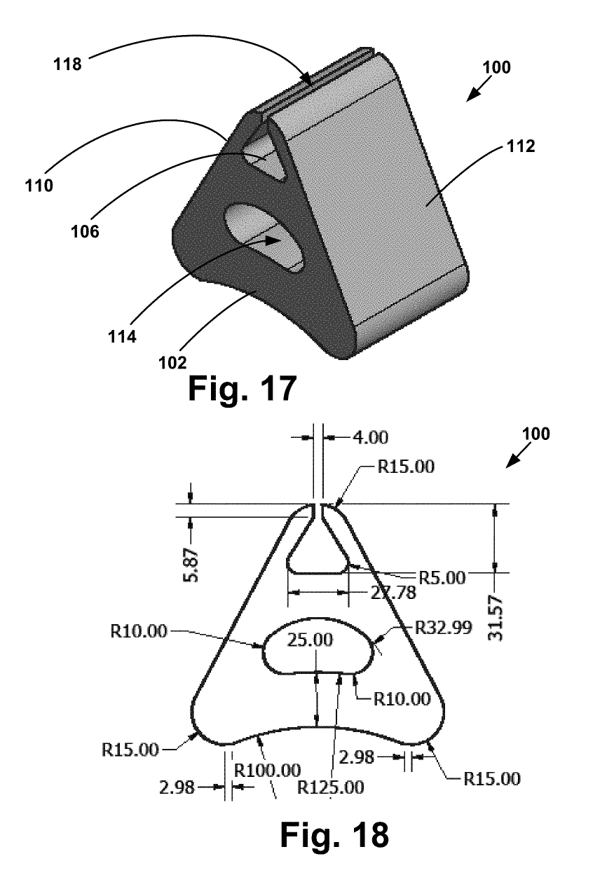

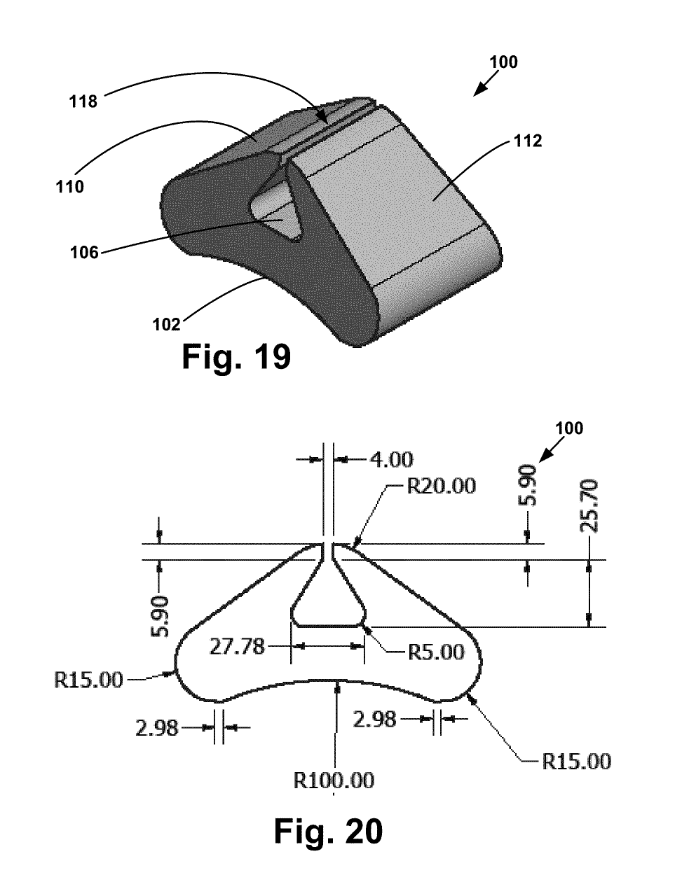

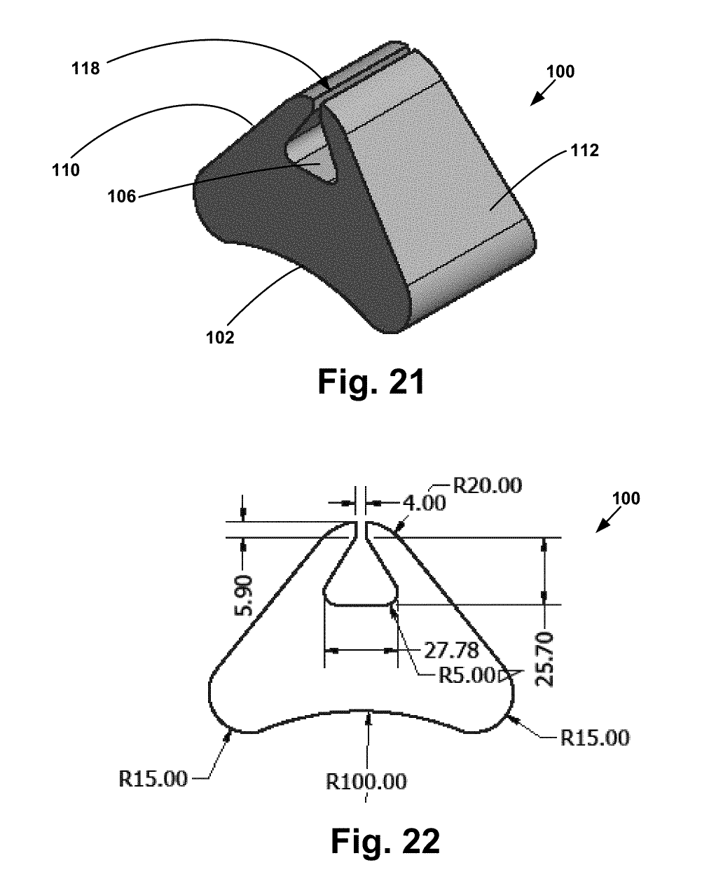

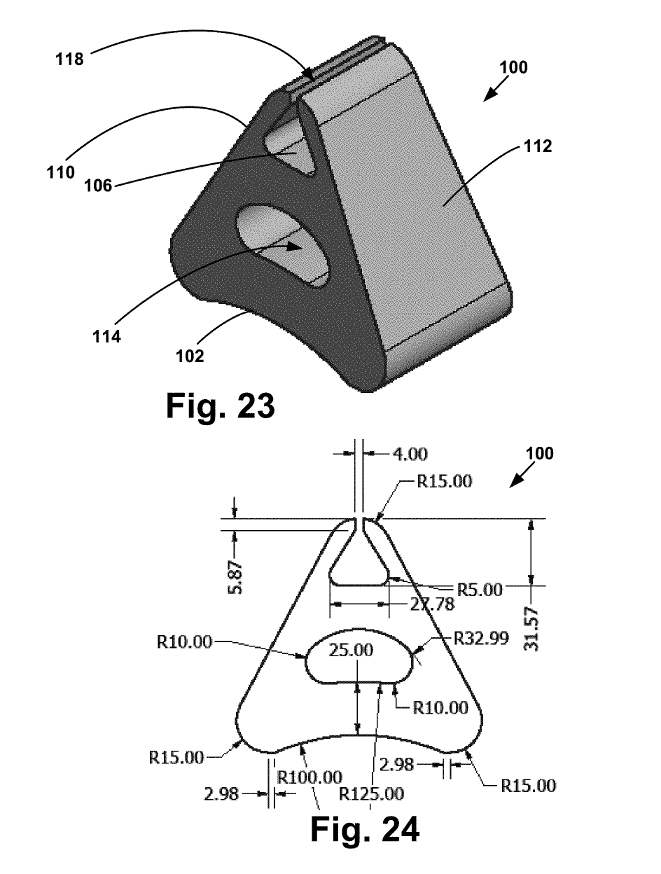

Thus, as shown in FIG. 1, the device 100 may be triangular in shape. In particular, the device 100 may include (i) a concave surface 102 at a first end 104 of the device 100, (ii) a conduit 106 at a second end 108 of the device, (iii) a pair of side surfaces 110, 112 extending inwardly at an acute angle from the first end 104 of the device 100 to the second end 108 of the device 100. In one example, as shown in FIGS. 1, 2-8, 10-11, 17-18, and 23-24, the device 100 may also include a cutout 114 positioned between the first end 104 and the second end 108 of the device. In another examples, as shown in FIGS. 13-16 and 19-22, the device 100 does not include the cutout 114. The concave surface 102 may be shaped such to accommodate, for example, a shape of the chest of the patient while the device is in use. In addition, the edges 116 of the device 100 may be rounded to help minimize the risk of bruising the skin of the patient, as shown in FIGS. 3 and 4. In one example, the width of the device 100 may taper from the first end 104 to the second end 108, such that the width at the first end 104 is greater than the width at the second end 108, as shown in FIGS. 9 and 12.

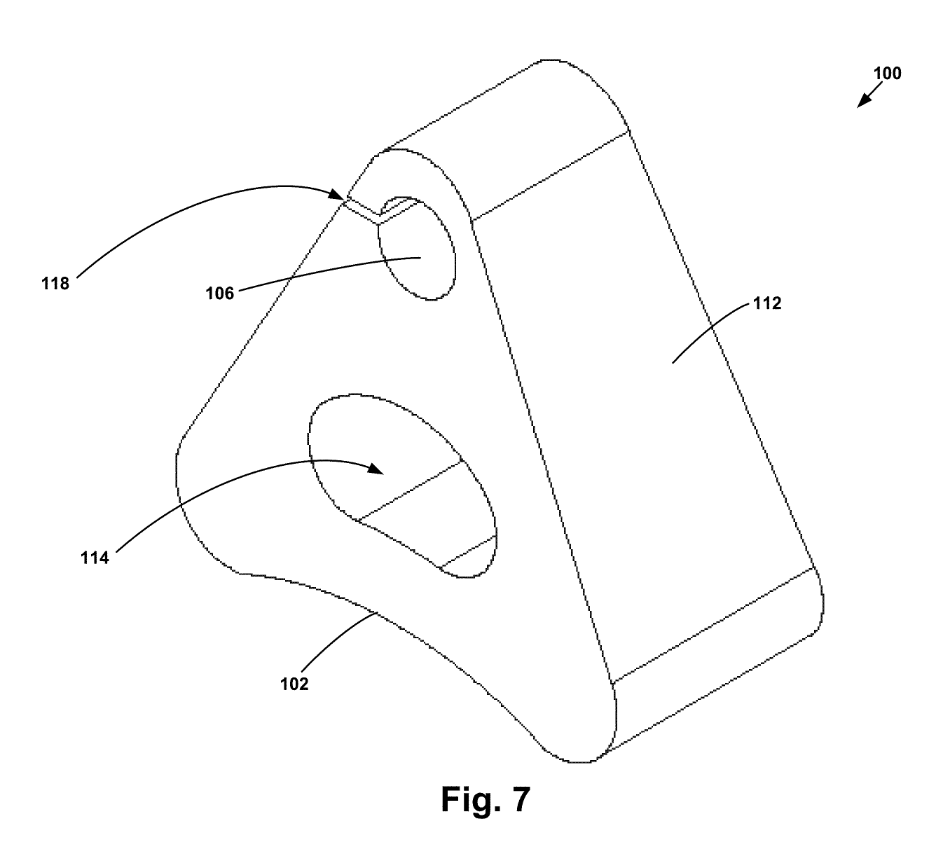

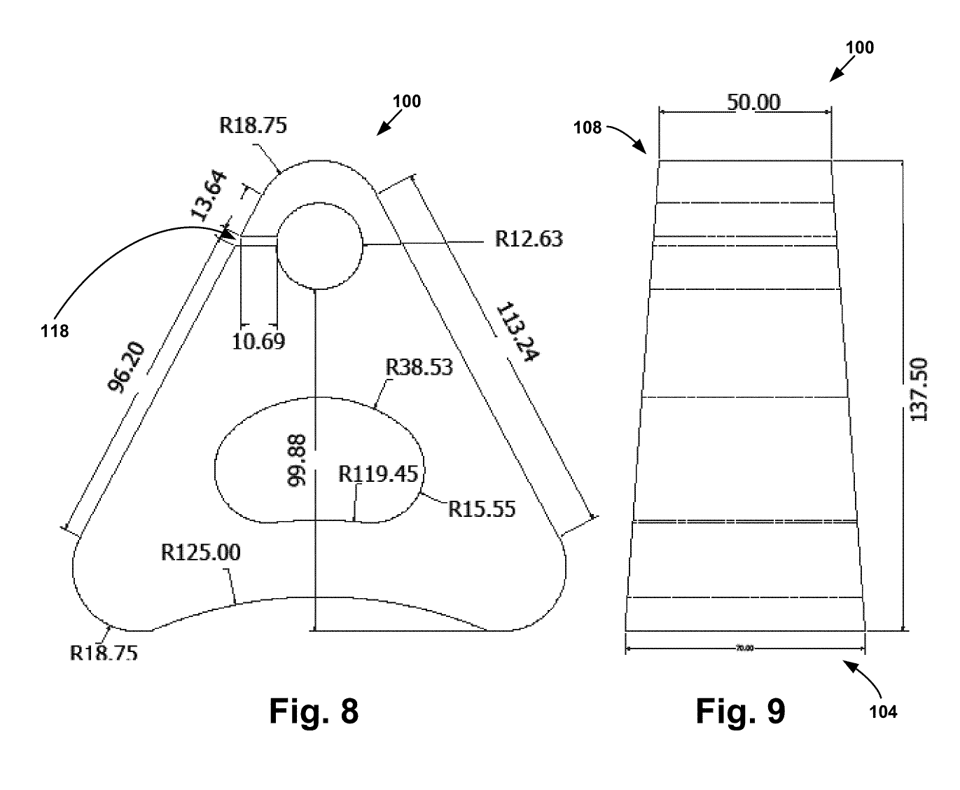

Further, as shown in the Figures, the device may include a slot 118 coupled to the conduit 106. The material of the device 100 may enable the slot 118 to open slightly when a tube is positioned therein, and then snap back to close the top of the conduit 106 once the tube is in position. In the example shown in FIGS. 1-6, 10-11, and 13-24, the slot 118 may be positioned at the top portion of the conduit 106. In another example, as shown in FIGS. 7 and 8, the slot 118 may be positioned on the side of the conduit 106. In addition, as shown in FIGS. 1 and 5-6, an interior surface of the conduit 106 of the device 100 may include one or more slits 120 that may be used to hold circuitry of varying diameters. In addition, the device 100 may further include one or more slits 122 in the pair of side surfaces 110, 112 and/or the cutout 114, as shown in FIG. 1. These slits 122 may be used to hold blood tubes, IV tubes, or any other lines that may be used during a given procedure.

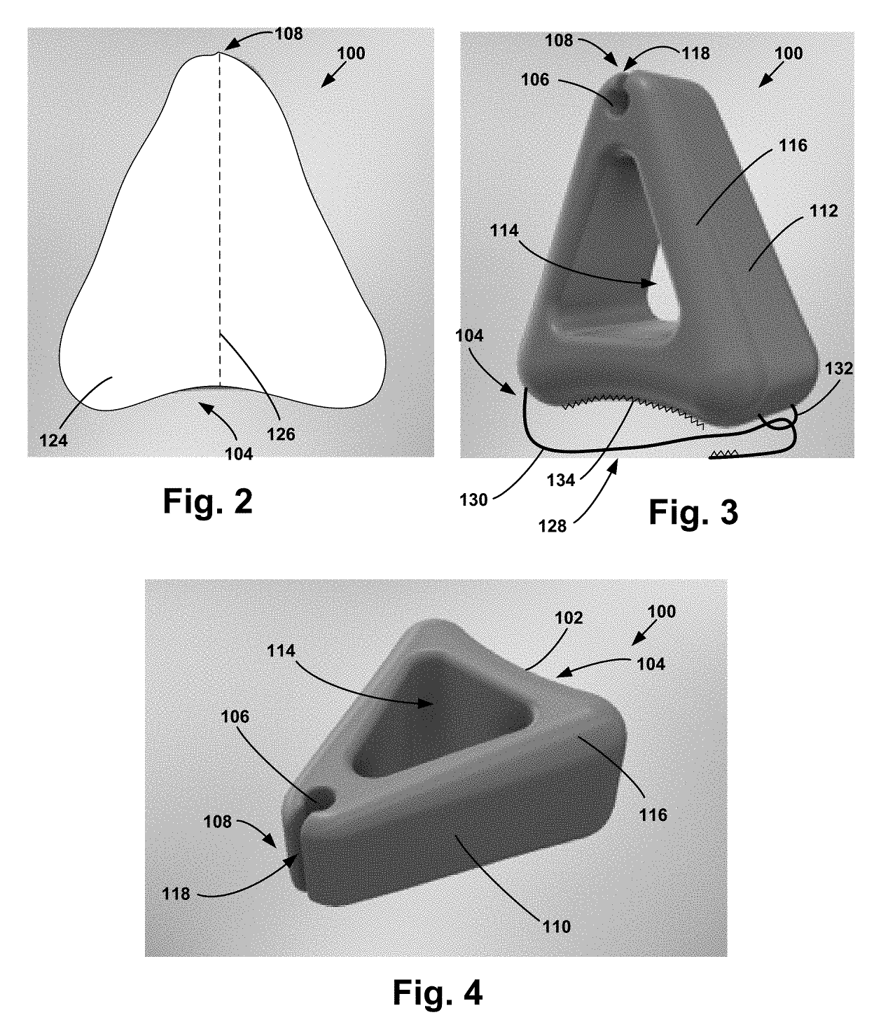

In one embodiment, as shown in FIG. 2, the device 100 may include a removable, washable cover 124 to promote hygiene and added comfort. The removable, washable cover 124 may be placed over the device 100 and secured by a zipper 126 or other securing means. In such an embodiment, the device 100 may be comprised of varying resilient materials including but not limited to memory foams, plastic foams, popped polystyrene, and other natural and man-made fibers, while the cover 124 may comprise a plastic or otherwise non-porous, waterproof, and sterile material.

In other embodiments, the device 100 may be used without the removable, washable cover 124. In such an example, the device 100 may comprise, for example, a non-porous foam material, such as closed-cell polyurethane or closed-cell polyethylene foam. In one particular example, the material of the device 100 may comprise a crosslinked polyethylene foam. Other materials are possible as well.

In some embodiments, the side surfaces 110, 112 of the device 100 may comprise a different material than the other components of the device 100. As a specific example, the side surfaces 110, 112 may comprise a polyurethane foam of lower density than a polyurethane foam that makes up the concave surface 102 at the first end 104 of the device 100. As such, the side surfaces 110, 112 may comprise a more rigid material than the concave surface 102. Such an embodiment may help provide sufficient rigidity for supporting the weight of the endotracheal or tracheostomy tube and corresponding ventilator circuit with minimum risk for bruising the skin of the patient. Other examples are possible as well.

As shown in FIG. 3, the device 100 may further include a coupling mechanism 128 configured to position the device 100 on the chest of the patient. In one example, the coupling mechanism 128 comprises a strap 130 that may be fixed to a first side of the first end 104 of the device 100. In such an example, the device 100 may also include a loop 132 at a second side of the first end 104 of the device 100. The strap 130 may be a hook fastener strap including a first potion with a plurality of hooks and a second portion including a plurality of loops. In such an embodiment, the strap 130 may be fed around the back of the patient, through the loop 132 on the second side of the first end 104 of the device 100, and then the first portion of the strap 130 may be attached to the second portion of the strap 130 on the back of the patient. In another example, the concave surface 102 at the first end 104 of the device 100 may include a hook fastener 134 that is configured to attach directly to the clothing of the patient, such as a surgical gown. A representative example of a hook fastener is Velco® In yet another example, the concave surface 102 at the first end 104 of the device 100 may include adhesive tape or glue dots that is configured to attach directly to the clothing of the patient. Any other suitable manner may be used to secure the device 100 to the patient. These embodiments may help secure the device 100 to the patient when the device 100 is in use. Other coupling mechanisms 128 are possible as well. In another example, the device 100 may simply rest on the chest of the patient when in use.

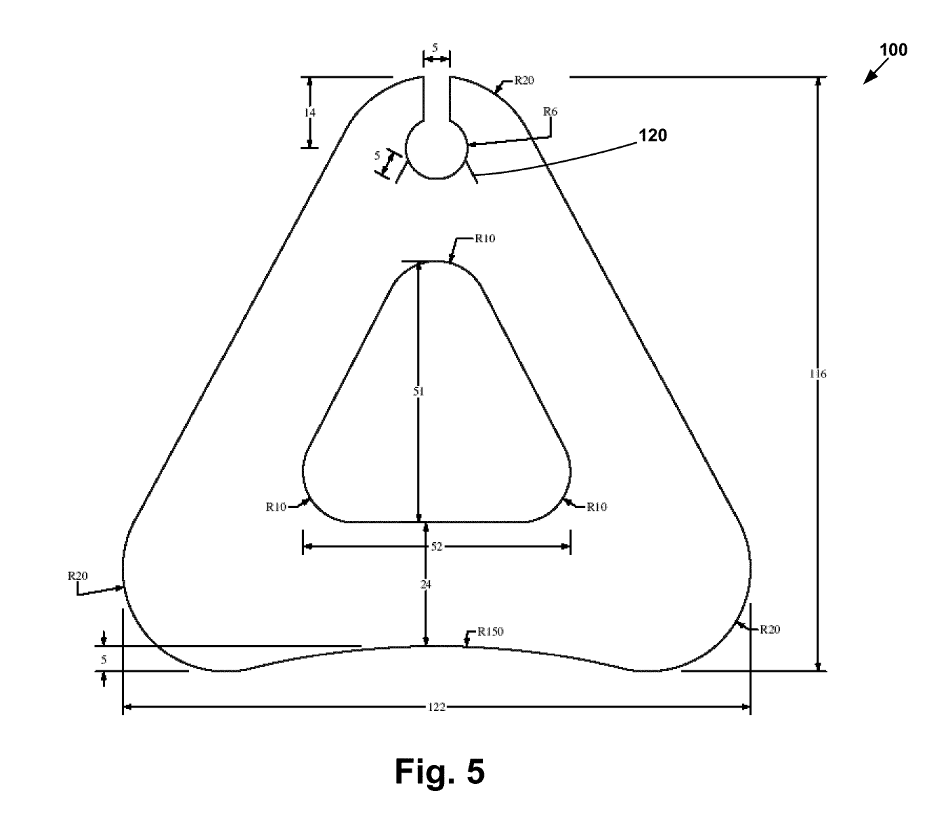

FIGS. 5-24 illustrate several versions of the device 100. The measurements shown in FIGS. 5-24 are in mm. The particular dimensions shown in FIGS. 5-24 are for illustrative purposes only, and are not meant to limit the scope of the device 100 to those particular dimensions. For example, the width of the device 100 may range from about 60 mm to about 175 mm, the height of the device 100 may range from about 50 mm to about 165 mm, and the depth of the device 100 may range from about 30 mm to about 110 mm.

In addition, while several sizes are shown in the figures, any sized device is contemplated depending on the particular use case. For example, the device 100 may include a small, medium, large, or extra-large size depending on the size of the patient. Further, the device 100 may include additional size ranges for children. Further still, the device 100 may include a different contour of the concave surface 102 and/or additional sizes for women compared to men. Other example sizes are possible as well. In one particular example, a large (e.g., taller) version of the device 100 may be used for endotracheal tubes, while a small (e.g., shorter) version of the device 100 may be used for tracheostomy tubes.

In use, the device 100 may be positioned on the chest of the patient. The device 100 may be strapped to the patient, or simply rest on the chest of the patient, as discussed above. Once the device 100 is in place, the endotracheal tube or tracheostomy tube may be passed through the conduit 106 at the second end 108 of the device 100. The second end 108 of the device 100 may include a slot 118 coupled to the conduit 106, as shown in FIGS. 1-8, 10-11, and 13-24. The material of the device 100 may enable the slot 118 to open slightly when the tube is positioned therein, and then snap back to close the conduit 106 once the tube is in position. Such a configuration may help secure the tube in the conduit 106. In addition, as discussed above, the conduit 106 may include one or more slits 120. The one or more slits 120 may be used to hold circuitry of the endotracheal tube or tracheostomy tube of varying diameters. In particular, the slits 120 may enable the conduit 106 to expand to receive ventilator circuits of varying diameters. Further, the device 100 may include a cutout 114 positioned between the first end 104 and the second end 108 of the device 100, through which additional components or circuitry may be passed. In addition, the device 100 may include one or more additional slits 122 in the pair of side surfaces 110, 112 and/or the cutout 114. These additional slits 122 may be used to hold blood tubes, IV tubes, or any other lines that may be used during a given procedure.

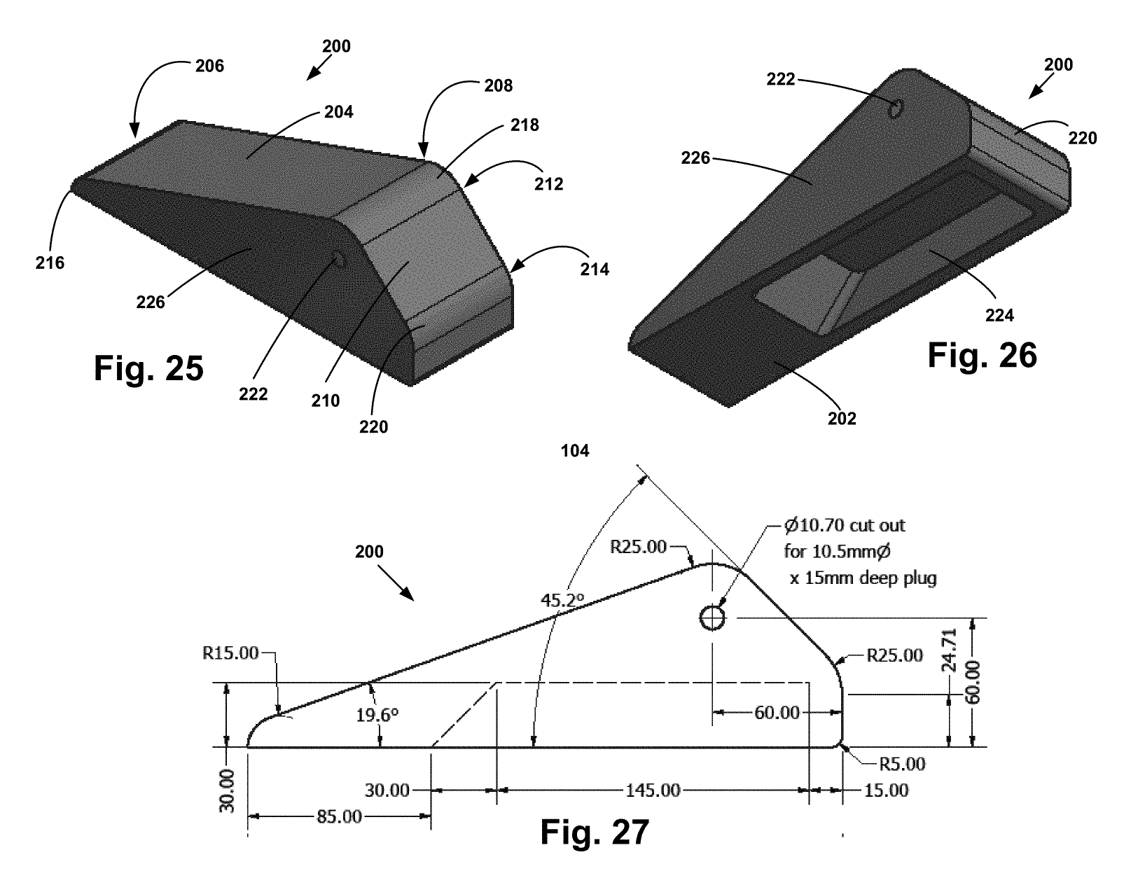

In another example, as shown in FIGS. 25-27, another device 200 is disclosed. The device 200 shown in FIGS. 25-27 may include (i) a bottom surface 202, (ii) a first angled surface 204 having a first end 206 and a second end 208, and (iii) a second angled surface 210 having a first end 212 and a second end 214. In one example, as shown in FIG. 25, the device 200 may include a first rounded surface 216 coupling the bottom surface 202 to the first end 206 of the first angled surface 204, a second rounded surface 218 coupling the second end 208 of the first angled surface 204 to the first end 212 of the second angled surface 210, and a third rounded surface 220 coupling the second end 214 of the second angled surface 210 to the bottom surface 202. The device 200 may further include a through-hole 222 having a longitudinal axis substantially parallel to the longitudinal axis of the second rounded surface 210.

The bottom surface 202 of the device 200 may be substantially flat, or may include a cavity cutout 224 as shown in FIG. 26. The first angled surface 204 may have a first angle with respect to the bottom surface 202, and the second angled surface 210 may have a second angle with respect to the bottom surface 202. In one example, the first angle is less than the second angle. Further, a length of the first angled surface 204 may be longer than a length of the second angled surface 210.

The device 200 of FIGS. 25-27 may be comprised of varying resilient materials including but not limited to other memory foams, plastic foams, popped polystyrene, closed-cell polyurethane, closed-cell polyethylene foam, a crosslinked polyethylene foam, and other natural and man-made fibers. In some embodiments, the side surfaces 226 of the device 200 may comprise a different material than the angled surfaces 204, 210 of the device 200. As a specific example, the side surfaces 226 may comprise a polyurethane foam of lower density than a polyurethane foam that makes up the angled surfaces 204, 210 of the device. Such an embodiment may help provide sufficient rigidity for supporting the weight of an arm of a user with minimum risk for bruising the skin of the patient. Other examples are possible as well.

In use, the device 200 of FIGS. 25-27 may be positioned on a flat surface, such as a table. A user may then position his or her arm on the first angled surface 204, thereby exposing the radial artery of the user. The first angled surface 204 may thereby position the user's arm in the ideal position for a practitioner to insert a needle into their radial artery.

It should be understood that arrangements described herein are for purposes of example only. As such, those skilled in the art will appreciate that other arrangements and other elements (e.g. machines, interfaces, functions, orders, and groupings of functions, etc.) can be used instead, and some elements may be omitted altogether according to the desired results. Further, many of the elements that are described are functional entities that may be implemented as discrete or distributed components or in conjunction with other components, in any suitable combination and location, or other structural elements described as independent structures may be combined.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims, along with the full scope of equivalents to which such claims are entitled. It is also to be understood that the terminology used herein is for the purpose of describing particular embodiments only, and is not intended to be limiting.

Since many modifications, variations, and changes in detail can be made to the described example, it is intended that all matters in the preceding description and shown in the accompanying figures be interpreted as illustrative and not in a limiting sense. Further, it is intended to be understood that the following clauses (and any combination of the clauses) further describe aspects of the present description.

We claim:

1. A device, comprising:
a concave surface at a first end of the device;
a conduit at a second end of the device; and
a pair of side surfaces extending inwardly at an acute angle from the first end of the device to the second end of the device.

2. The device of claim 1, wherein the edges of the first end of the device are rounded.

3. The device of claim 1, wherein a width of the device tapers from the first end to the second end, such that a width at the first end is greater than a width at the second end.

4. The device of claim 1, further comprising a slot coupled to the conduit, wherein the slot is configured to open when a tube is positioned therein, and then snap back to close the conduit once the tube is in position in the conduit.

5. The device of claim 1, further comprising a cutout positioned between the first end and the second end of the device.

6. The device of claim 5, further comprising one or more slits positioned in one or more of an interior surface of the conduit, an interior surface of the cutout, or the pair of side surfaces.

7. The device of claim 1, further comprising a removable, washable cover.

8. The device of claim 7, wherein the cover comprises a plastic or otherwise non-porous, waterproof, and sterile material, and wherein the device comprises memory foam, plastic foam, or popped polystyrene.

9. The device of claim 1, the device comprises a non-porous foam material, such as closed-cell polyurethane or closed-cell polyethylene foam, or a crosslinked polyethylene foam.

10. The device of claim 1, wherein the pair of side surfaces comprise a first material, and wherein the concave surface comprises a second material that is different than the first material.

11. The device of claim 10, wherein the first material is more rigid than the second material.

12. The device of claim 1, further comprising a coupling mechanism configured to position the device on the chest of the patient.

13. The device of claim 12, wherein the coupling mechanism comprises:
a strap fixed to a first side of the first end of the device; and
a loop at a second side of the first end of the device.

14. The device of claim 12, wherein the coupling mechanism comprises a hook fastener on at least a portion of the concave surface.

15. The device of claim 1, wherein a width of the device ranges from about 60 mm to about 175 mm, the height of the device ranges from about 50 mm to about 165 mm, and the depth of the device ranges from about 30 mm to about 110 mm.

16. A method, comprising:
positioning the device of claim 1 on a chest of a subject; and
passing an endotracheal tube or a tracheostomy tube through the conduit at the second end of the device and into the subject.

17. A device, comprising:
a bottom surface;
a first angled surface having a first end and a second end;
a second angled surface having a first end and a second end;
a first rounded surface coupling the bottom surface to the first end of the first angled surface;
a second rounded surface coupling the second end of the first angled surface to the first end of the second angled surface; and
a third rounded surface coupling the second end of the second angled surface to the bottom surface.

18. The device of claim 17, further comprising a through-hole having a longitudinal axis substantially parallel to a longitudinal axis of the second rounded surface.

19. The device of claim 17, wherein the first angled surface has a first angle with respect to the bottom surface, and wherein the second angled surface has a second angle with respect to the bottom surface that is greater than the first angle.

20. The device of claim 17, wherein a pair of side surfaces of the device comprise a first material, wherein the first and second angled surfaces comprise a second material that is different than the first material, and wherein the first material is more rigid than the second material.
